(19) United States
(12) Plant Patent Application Publication
(10) Pub. No.: US 2013/0239269 P1
(43) Pub. Date: Sep. 12, 2013

(54) Nerium oleander plant-Turner's 7-679

(50) ;

(71) Applicant: Ted L. Turner, SR., Corpus Christi, TX (US)

(72) Inventor: Ted L. Turner, SR., Corpus Christi, TX (US)

(21) Appl. No.: 13/385,861

(22) Filed: Mar. 12, 2012

(51) Int. Cl. A01H 5/00

(52) U.S. Cl. PLT/233

(57) ABSTRACT

A Nerium oleander plant which has an upright, slightly weeping branches when in full bloom, being particularly characterized by long blooming periods and a unique color of it's inflorescence and it's habit of growth, the flowers being a multicolor of delicate pink with a light yellow eye and the corona having long eyelash like appendages.

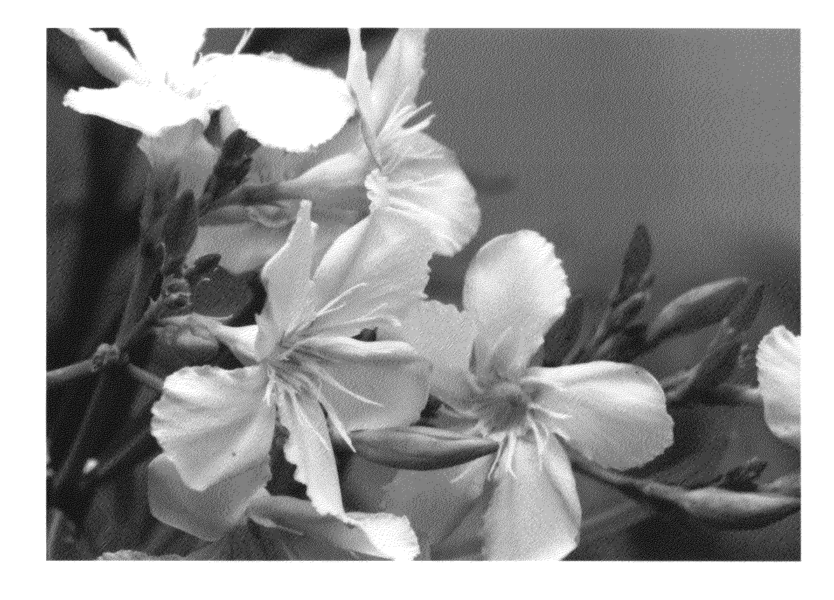

The present invention relates to a new and distinct variety of Nerium oleander which was originated by me as a seedling by selection and crossing. I crossed a Nerium oleander “Turner's Shari D’ with Nerium oleander ‘Turner's Trey Boy.’ ‘Turner's Shari D’ is the subject of U.S. Plant Pat. No. 5,378 issued Dec. 25, 1984, to Ted L. Turner, Sr., and this plant is an intermediate to full size plant with flowers which are soft buff yellow with tinges of pink. ‘Turner's Trey Boy’ is the subject of U.S. Plant Pat. No. 16,660 issued Jun. 13, 2006 to Ted L. Turner, Sr., and this plant is a petite size plant with flowers which are dusty rose. However, in making my crosses, I observed that pollination could have been accomplished by bees from unknown plants. Thus unknown parentage could have been introduced by the bees even though I did not purposely introduce any parentage other than the ‘Turner's Shari D’ and ‘Turner's Trey Boy.’ The Nerium oleander of my new variety differs from the ‘Turner's Shari D’ and the ‘Turner's Trey Boy’ mentioned above mainly in the growth habit and the color and shape of the flowers. The growth habit of my new variety is of intermediate size with flowers which are blush pink in color. The flower petals have marked undulate edges. The interior of the corolla is prominently ciliate with the aspect of eyelashes.

From the group of seedlings I observed a plant, being the plant claimed herein, have a new and distinct color of flowers, and from cuttings of such plant I was able to asexually reproduce plants having the same characteristics and flower color as the original seedling. The plant observed for this patent application has been grown in a greenhouse, was in a seven gallon container, and was about five years old. All of the descendant plants showed the same characteristics as the original seedling, and as a result of extensive observations and tests which are not described in full herein for sake of brevity, it is my opinion and I am convinced that my new plant is a new variety of Nerium oleander which is distinguished from all other varieties of which I am aware as evidenced by the following unique combination of principal characteristics which are outstanding therein:

(1) An up-right, slightly weeping habit of fairly rapid growth and of intermediate size making it especially suitable for containers; as landscape wind breaks; and as screening for large buildings;

(2) An ability to be asexually reproduced;

(3) An ability to flower substantially throughout the year with long continuous blooming periods;

(4) The ability to bloom in full sun or in partial shade, such as morning sun only;

(5) The ability to be a good greenhouse bloomer and bloom year round in a greenhouse that is heated in the winter;

(6) The ability to initially grow and flower in pots as small as about 10 centimeters in diameter, although the plant should be placed in a larger pot of at least 60 centimeters to obtain larger growth and more blossoms;

(7) The ability to produce a flower having a blush pink color with areas of light pink at the edges of the petals as well as areas of darker pink in the corona;

(8) The ability to produce a plant having flowers arranged in cymes or clusters with appendages of the corona that resemble eyelashes;

(9) The ability to grow in windy conditions.

Asexual reproductions of my new variety as by cuttings, show that the foregoing characteristics and distinctions come true to form and are established and transmitted through succeeding propagations. The ongoing planned breeding program which resulted in the plant described herein, and the initial asexual reproduction of this plant has taken place in a nursery in Corpus Christi, Nueces County, Tex.

The accompanying FIGURE is a photograph of typical flowers on the plant of my new variety more clearly illustrating the colors and features present on each flower. The true color of the foliage and flowers is described in the following description and is depicted in the FIGURE which depicts the color as nearly as true as is reasonably possible in a color illustration of this type.

The following is a detailed description of my new variety of Nerium oleander, which color numbers are in accordance with the Pantone Matching system of colors for printing inks, Pantone Dolor Formula Guide 747 XR, 1997-1998, published by Pantone, Inc. Terms used to describe colors are those of ordinary significance.
The plant:
Growth habit: Upright, medium size; the height of an unpruned mature plant will be from about 2.1 to 3 meters, but has not yet been determined; fast growing in shady areas and slower in full sun. The plant grows upright with its height generally twice the size of its width. The plant is about the same height as that of Turner's Shari D' and much larger than ‘Turner's Trey Boy’ referenced above.
Hardiness: Adapted to seaside planting as it tolerates soil with relatively high salt content; tolerates droughts; will not withstand prolonged and severe freezing weather; most suitable for the Southern United States from California to Florida, in the areas known as Zones 9 and 10; withstands heat and light and grows either in full sun or in partial shade (up to 50% shade) such as morning sun only; will bloom in small pots as small as about 10 centimeters in diameter, although the plant should be transferred to larger pots for larger growth.
Branches: The plant develops central leaders or trunk, with main branches and branchlets slightly drooping. New growth of branches is light to medium green color, PMS No. 390, changing to light greenish brown in color, PMS No. 125, as branches mature. The old or matured wood is a pale brown, PMS No. 456.
Blooming period: With outside conditions, blooms off and on substantially the entire year, being in bloom about 80% of the time. The plant will bloom substantially all the time in a greenhouse with proper feeding, watering, and additional heat in winter months.
The foliage:
Type: Broadleaf evergreen; numerous; petioled; grows in a whorl with three leaves in each whorl.
Shape: Linear-lanceolate, with entire margins. Apex is more or less acuminate and slightly non-symmetrical, and base is acute.
Petioles: Length, from about 3 to 6 mm; color pale green, PMS no. 390.
Leaf size: Length of mature leaf from 108 mm to 142 mm; the width of a mature leaf about 18 to 21 mm. Size of leaves varies according to light or dark days at the time the leaf is produced, with larger leaves being produced under cloudy conditions than under sunny conditions.
Venation: Midrib on under surface prominent and readily apparent and is pale green color, PMS No. 397; on upper surface midrib is clearly visible and slightly recessed, and is pale green, PMS No. 3975; lateral veins are not readily apparent on upper surface, but under surface contains numerous, delicate, almost parallel lateral veins which are readily apparent, the lateral veins being substantially perpendicular to midrib.
Leaf color: — Mature leaves: upper surface — dark green, PMS No. 351; under surface lighter in color than upper surface, being a medium green, PMS No. 370.
New leaves: The colors of new leaves, PMS No. 368, are lighter and brighter in color than matures leaves, PMS No. 351.
Leaf texture: Tough, leathery; upper surface — non-glossy; lower surface — dull.
The inflorescence:
Position and abundance: Flowers cluster at twig or branch ends in terminal cymes, with cymes appearing at various positions from the lower part to the upper part of the plant. Each cyme is tightly clustered with generally about 9 to 12 flowers each, although some cymes may contain more or less flowers.
Form: Single, regular; pediceled petals united in a sympetalous corolla; salver form; tube spreads into five limbs or lobes; each flower is about 50 to 55 mm across; corona conspicuous at junction of tube and spreading limb, corona about 10 to 12 mm in diameter and a depth of about 10 mm which is much shorter than spreading limbs, the corona having five crown like appendages, each appendage being 6 to 10 mm long and 1 to 2 mm wide, giving an appearance of eyelashes.
Buds: Limbs convolute in the bud, obliquely apiculate, the folds twisting counterclockwise when viewing down into the tip of the bud, such the limbs of the flower twist slightly clockwise when viewing down into the flower.
Calyx: Of 5 persistent sepals, imbricate in the bud, lanceolate, acuminate, about 5 mm long. The sepals have a relatively smooth surface texture. The calyx and sepals are light green, PMS No. 345.
Stamen: 5 stamens; filaments partly adnate to corolla tube; anthers with 2 basal ails, apex long — attenuate, hairy. The stamens are about 5 to 7 mm in length, with a medium yellow color, PMS No. 468.
Pistil: The style portion of the pistil is pale yellow in color, PMS No. 468, with outer end of stigma portion being pale yellow color, and with the pistil being approximately 4 mm in length.
Color of flower: Limbs or petals of the corolla in new flowers are a light pink, PMS No. 196, on one side of petal with a darker pink, PMS No. 197, on the other side. The corona is pale yellow, being a pale version of PMS No. 461. The edge of the petals are slightly ruffled. The outside of the corolla tube of each flower is a delicate pink, PMS No. 197, with areas of golden yellow, PMS No. 459.
Fragrance: The flowers have no appreciable fragrance.
Color of buds: Dark pink PMS No. 688 with light yellow blending into the pink at base of bud, PMS No. 1345. To further describe my new variety of Nerium oleander, it is very similar in height to the other well-known medium size Nerium oleanders which grow in an upright manner, except for the color of the free blooming single flower of soft pink shade, and the blossom clusters are extremely large with corona appendages that resemble eyelashes.
Variety name: The proposed variety name of my new plant is Nerium oleander ‘Turner's 7-679’.

I claim:

1. A new and distinct variety of medium size Nerium oleander plant substantially as shown and described, having an upright habit of growth with slightly weeping branches when in full bloom, making it especially suitable for large containers and hedges, having an ability to be asexually reproduced, having an ability to flower off and on throughout the entire year with long continuous blooming periods, having the ability to grow in full sun or in partial shade, being an excellent greenhouse plant, and being particularly characterized by the unique color of its inflorescence, and having terminal cymes of flowers which are a blush pink color with pronounced appendages on the corona of the flowers resembling extremely long eyelashes.
